(19) United States
(12) Patent Application Publication
(10) Pub. No.: US 2009/0264933 A1
(43) Pub. Date: Oct. 22, 2009

(54) ANCHORS FOR SECURING A ROD TO A VERTEBRAL MEMBER

(75) Inventors: Thomas A. CARLS, Memphis, TN (US); Michael S. Veldman, Memphis, TN (US); Jonathan M. Dewey, Sunnyvale, CA (US); Christopher M. Patterson, Olive Branch, MS (US); Todd Lanman, Rolling Hills, CA (US)

Correspondence Address: MEDTRONIC;Attn: Noreen Johnson - IP Legal Department, 2600 Sofamor Danek Drive, MEMPHIS, TN, 38132, US

(73) Assignee: Warsaw Orthopedic, Inc., Warsaw, IN (US)

(21) Appl. No.: 12/107,110

(22) Filed: Apr. 22, 2008

(51) Int. Cl. A61B 17/70; A61B 17/04

(52) U.S. Cl. 606/264; 606/305; 606/308

(57) ABSTRACT

Anchors to secure a rod to a vertebral member. The anchors may include a fastener with a distal end adapted to be connected to the vertebral member and a proximal end. A receiver may be operatively connected to the proximal end of the fastener. The receiver may include a channel sized to receive the rod. The receiver may further include at least one convex section that extends inward towards a longitudinal axis of the rod. The receiver may be formed in a unitary one-piece construction. The anchor may also include a securing member that connects to the receiver and may include a lower edge that extends into the channel and may contact the rod.

### BACKGROUND

The present application relates to anchors for securing a rod to a vertebral member, and more particularly to anchors with a convex surface that contacts the rod for dynamic spinal stabilization.

The spine is divided into four regions comprising the cervical, thoracic, lumbar, and sacrococcygeal regions. The cervical region includes the top seven vertebrae identified as C1-C7. The thoracic region includes the next twelve vertebrae identified as T1-T12. The lumbar region includes five vertebrae L1-L5. The sacrococcygeal region includes nine fused vertebrae that form the sacrum and the coccyx. The vertebrae of the spine are aligned in a curved configuration that includes a cervical curve, thoracic curve, and lumbosacral curve.

Rods may be implanted to support and position vertebral members in one or more of these regions. The rods extend along a section of the spine and are connected to the vertebral members with one or more anchors. The anchors are typically screwed into the posterior portions of the vertebral member and pass through the pedicles and a substantial portion of the vertebral bodies and therefore provide a fixed and durable connection. The spinal rods are then clamped to the anchors creating a rigid stabilization structure. In most situations, one such structure is provided on each lateral side of the spine.

While such structures hold the vertebral members correctly positioned relative to each other, they tend to considerably stiffen the spine. This may significantly limit the patient's post-operative freedom of movement and/or may lead to undesirable loadings on nearby vertebral members. Accordingly, efforts have been made to develop stabilization approaches that can tolerate some movement, with the resulting systems typically referred to as dynamic spinal stabilization systems.

### SUMMARY

The present application is directed to anchors to secure a rod to a vertebral member. The anchors may include a fastener with a distal end adapted to be connected to the vertebral member and a proximal end. A receiver may be operatively connected to the proximal end of the fastener. The receiver may include a channel sized to receive the rod. The receiver may further include at least one convex section that extends inward towards a longitudinal axis of the rod. The receiver may be formed in a unitary one-piece construction. The anchor may also include a securing member that connects to the receiver and may include a lower edge that extends into the channel and may contact the rod.

Other aspects of various embodiments of the anchor are also disclosed in the following description. The various aspects may be used alone or in any combination, as is desired.

### BRIEF DESCRIPTION OF THE DRAWINGS

FIG. 1 is a side view of an anchor that connects a rod to a vertebral member according to one embodiment.

FIG. 2 is a side view of a dynamic spinal stabilization assembly secured to a spinal column with the spinal column in the neutral position according to one embodiment.

FIG. 3 is a side view of a dynamic spinal stabilization assembly secured to a spinal column with the spinal column in extension according to one embodiment.

FIG. 4 is a side view of a dynamic spinal stabilization assembly secured to a spinal column with the spinal column in flexion according to one embodiment.

FIG. 5A is a perspective view of a receiver and fastener according to one embodiment.

FIG. 5B is a sectional view cut along line 5B-5B of FIG. 5A of the receiver and the fastener.

FIG. 6 is a perspective view of an anchor with an offset receiver and a fastener according to one embodiment.

FIG. 7 is a sectional view of a channel of a receiver according to one embodiment.

FIG. 8 is a sectional view of a channel of a receiver according to one embodiment.

FIG. 9 is a sectional view of a channel of a receiver according to one embodiment.

FIG. 10 is a sectional view of a channel of a receiver according to one embodiment.

FIG. 11 is a side view of an anchor with a rod within a channel according to one embodiment.

FIG. 12 is a perspective view of an anchor with a rod according to one embodiment.

FIG. 13 is a schematic view of an anchor according to one embodiment.

### DETAILED DESCRIPTION

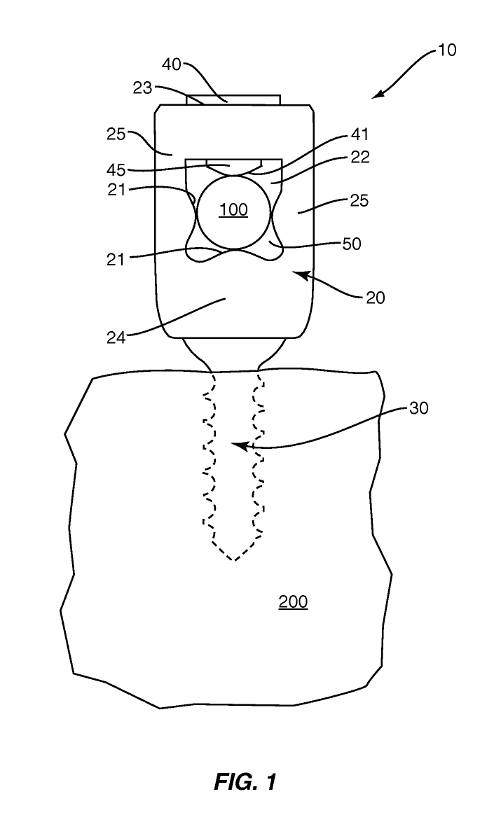

The present application is directed to anchors for connecting a rod to a vertebral member. FIG. 1 illustrates one embodiment of an anchor 10 that includes a receiver 20, fastener 30, and securing member 40 that connect a rod 100 to a vertebral member 200. The receiver 20 includes a channel 50 sized to receive the rod 100. The receiver 20 includes one or more convex surfaces 21 that contact against the rod 100. Each convex surface 21 provides for a limited contact area between the receiver 20 and the rod 100 to allow the rod 100 to translate relative to the anchor 10. The securing member 40 attaches to the receiver 20 and prevents the rod 100 from escaping from the channel 50. Securing member 40 may also include a convex surface 41 that contacts against the rod 100.

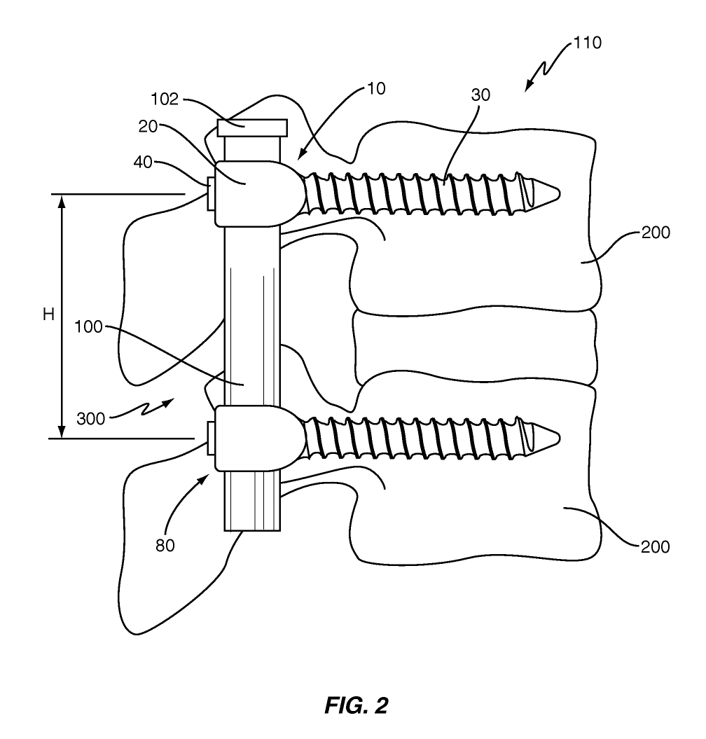

The convex surface 21 of the receiver 20 facilitates the rod 100 to move relative to the anchor 10 as opposed to be held in a fixed relative relationship. FIGS. 2, 3, and 4 illustrate one embodiment with the anchor 10 used as part of a dynamic stabilization assembly 300 that also includes the rod 100 and a second anchor 80. The second anchor 80 is fixed to the rod 100 and does not allow for movement between the second anchor 80 and rod 100. As illustrated in FIG. 2, anchor 10 and second anchor 80 are spaced from one another by distance H when the spinal column 110 is in an upright position without flexion or extension.

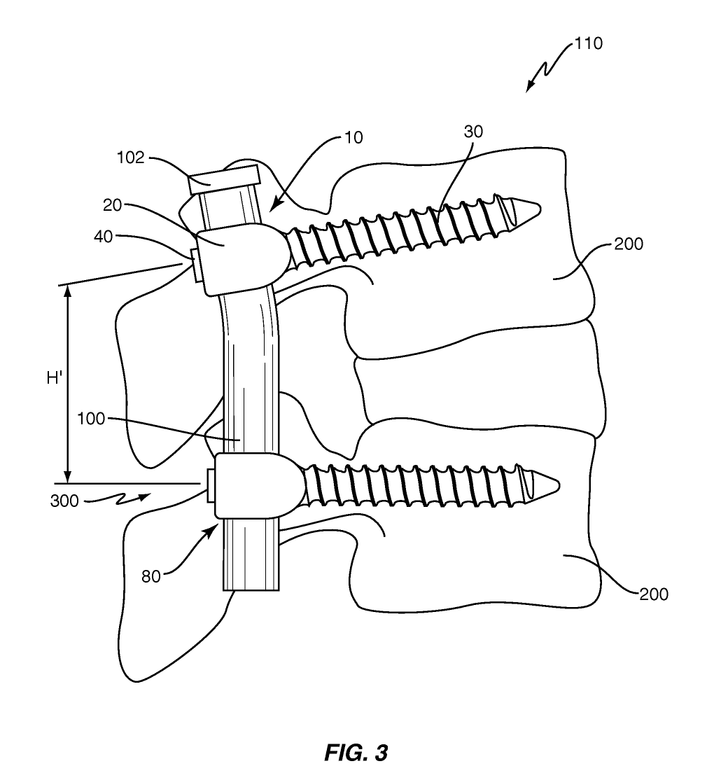

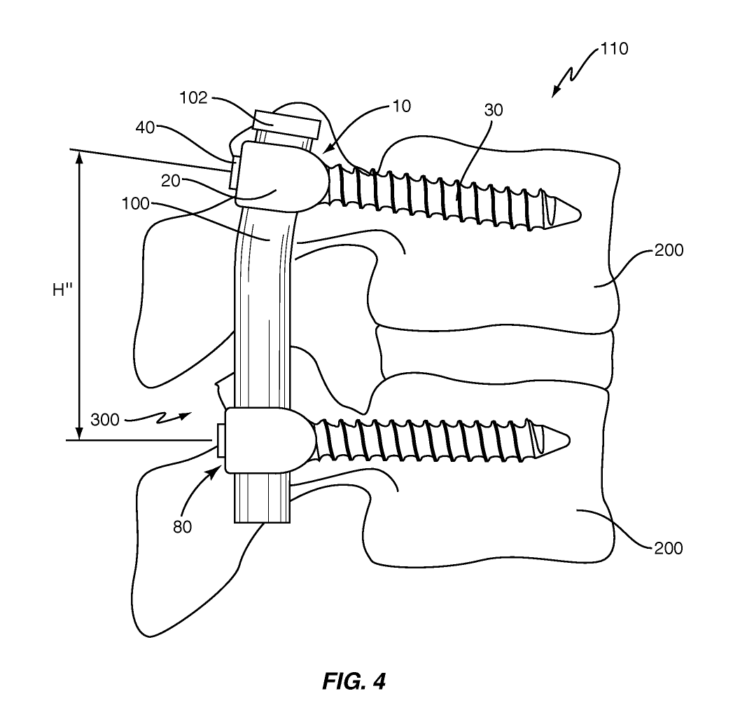

When the spinal column 110 undergoes extension as illustrated in FIG. 3, the anchor 10 will have a tendency to move inward along a length of the rod 100 thus shortening the distance to H′ between the anchors 10, 80. When the spinal column 110 undergoes flexion as illustrated in FIG. 4, the anchor 10 will have a tendency to move outward along the rod 100 and increase the distance to H″ between the anchors 10, 80.

The rod 100 may include one or more stops 102 positioned in proximity to the anchor 10. Stops 102 limit the relative movement between the rod 100 and anchor 10 to a predetermined amount. In the embodiments of FIGS. 2, 3, and 4, a stop 102 is positioned at an axial end of the rod 100 to prevent the anchor 10 from becoming disconnected. Bumpers (not illustrated) may also be positioned in proximity to the anchor 10 to elastically resist/dampen movement of the rod 100 relative to the anchor 10. Embodiments of dynamic stabilization assemblies are disclosed in U.S. patent application Ser. Nos. 11/668,746 and 11/668,792 each filed Jan. 30, 2007, and each herein incorporated by reference.

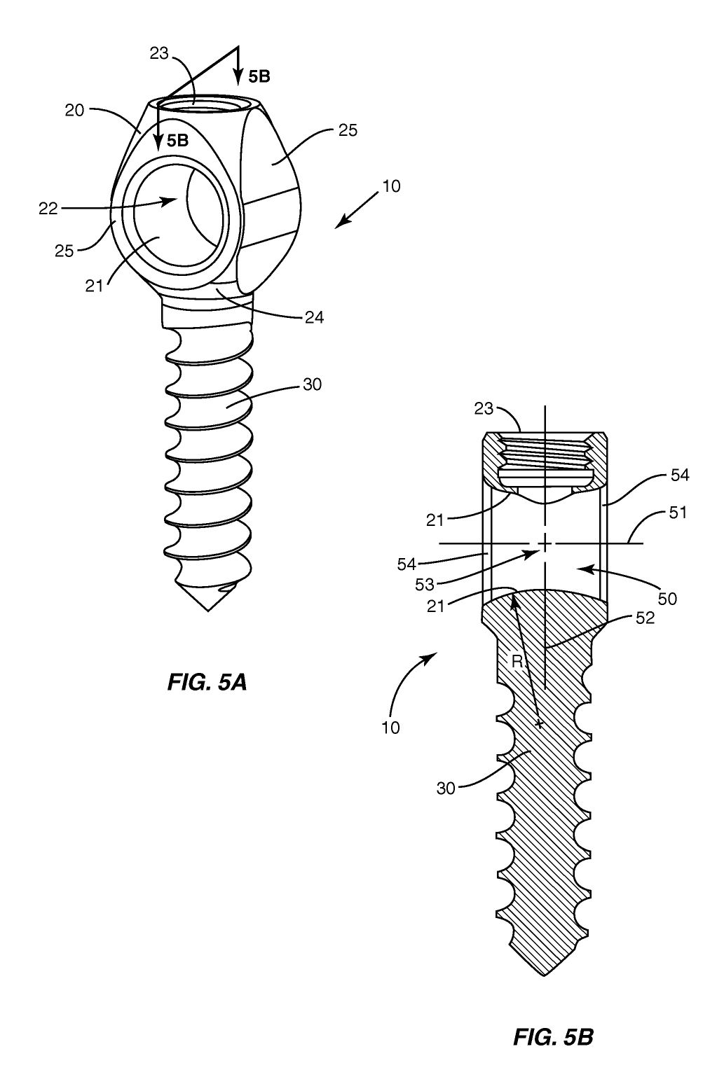

The receiver 20 may include a variety of different configurations depending upon the context of use. In one embodiment as illustrated in FIG. 1, receiver 20 includes a base 24 and side walls 25 that together form the channel 50. The side walls 25 are spaced apart and form an opening 23 configured to receive the securing member 40. FIGS. 5A and 5B illustrate another embodiment with the receiver 20 including a base 24 and a side wall 25 that extends completely around the channel 50. The opening 23 is formed through the side wall 25 and extends through to the channel 50.

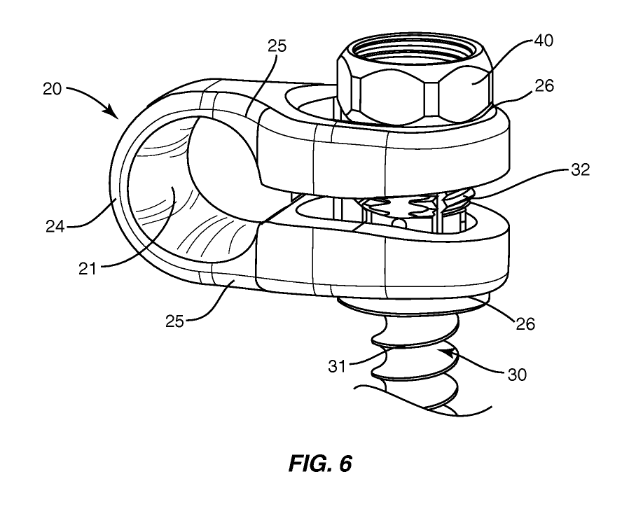

The embodiments illustrates in FIGS. 1-5B illustrate an in-line embodiment with the channel 50 being substantially aligned with a longitudinal axis of the fastener 30. FIG. 6 illustrates an offset embodiment with the receiver 20 configured such that the channel 50 is positioned away from the fastener 30. In FIG. 6, the receiver 20 includes a base 24 with opposing side walls 25. Each of the side walls 25 includes an opening 26 to receive the proximal end 32 of the fastener 30. The openings 26 may include an elongated shape to provide for lateral adjustment of the receiver 20 relative to the fastener 30. The securing member 40 is attached to the proximal end 32 and forces the side walls 25 causing the convex surface 21 to contact against the rod.

The receivers 20 in the various embodiments include one or more convex surfaces 21 that extend into the channel 50 from either the base 24 or side walls 25 to contact against the rod 100. FIG. 5B illustrates one embodiment with the channel 50 including a longitudinal axis 51. The channel 50 is non-cylindrical in that the convex surface 21 causes the channel 50 to taper outward from a midpoint 52. The profile of the channel 50 may be longitudinally divided for ease of reference into a medial section 53 centered on the midpoint 52 and respective end sections 54. As seen in FIG. 5B, the medial section 53 tapers both inward toward, and outward away from, axis 51, such that the convex surface 21 is disposed closer to axis 51 in the medial section 53 than the end sections 54.

In the embodiment of FIG. 5B, the convex surface 21 bows inward toward, or is convexly curved toward, axis 51, with a constant radius of curvature R. The convex surface 21 is substantially the same along the periphery of the channel 50 and is only interrupted by the opening 23. Further, the convex surface 21 extends along the substantially the entire width of the channel 50.

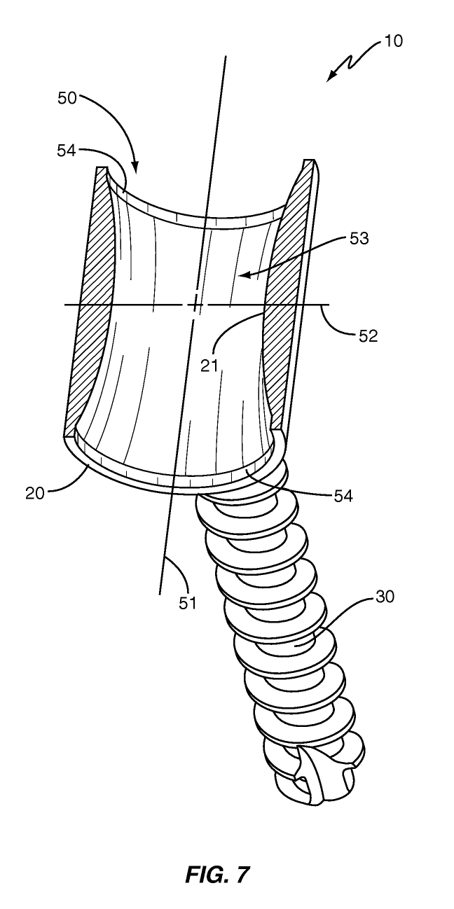

FIG. 7 illustrates another embodiment with the longitudinal profile of the channel 50 being relatively straight in the end sections 54, but bowed toward axis 51 in the medial section 53. FIG. 7 includes the receiver 20 as part of an in-line anchor 10.

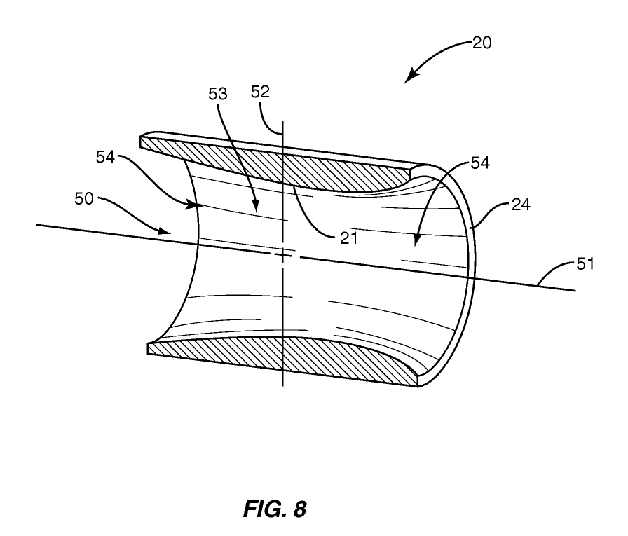

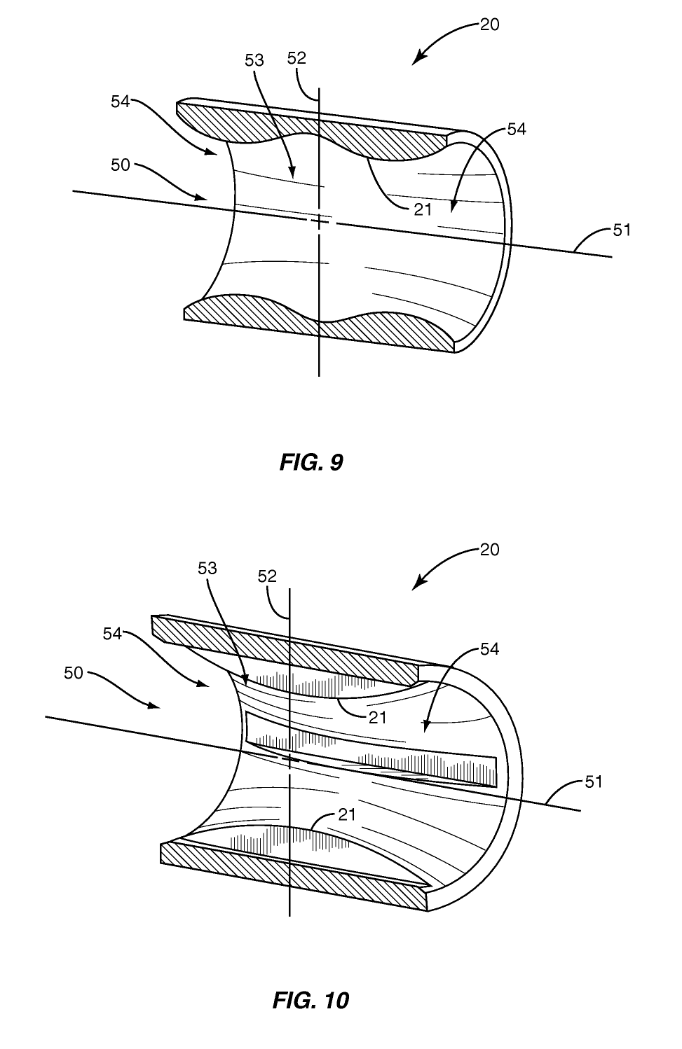

FIG. 8 includes an embodiment with the channel 50 such that the convex surface 21 approaches most closely to axis 51 at a point that is longitudinally off-center (i.e., axially offset along the longitudinal axis 51 from midpoint 52). The embodiment of FIG. 9 includes a profile of the channel 50 with two convex surfaces 21. The convex surfaces 21 approach most closely to axis 51 at two spaced apart points, creating two necked-down regions. The embodiment of FIG. 10 includes a plurality of discrete convex surfaces 21 spaced around the periphery of the channel 50. FIGS. 8-10 include embodiments with the convex surfaces 21 on the channels 50 of an offset receiver 20 with the side walls 25 of the receivers 20 removed for clarity. It is to be understood that the various aspects disclosed in the embodiments throughout this application may be used in the various different types of anchors 10. Further, the various aspects of the convex surfaces 21 may be combined as appropriate for different circumstances.

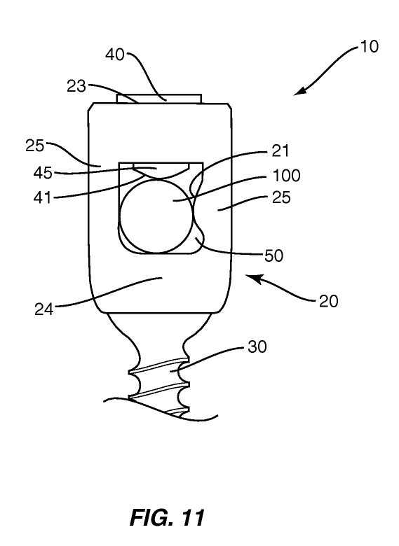

The convex surface 21 may extend around a majority of the channel 50. In another embodiment as illustrated in FIG. 11, the convex surface 21 is positioned along a discrete portion of the channel 50. FIG. 11 specifically includes the convex surface 21 extending inward into the channel 50 from one of the side walls 25. FIGS. 1 and 10 each include an embodiment with three discrete portions including convex surfaces 21. In embodiments with multiple convex sections 21, the sections may include the same or different curvatures and lengths.

The receiver 20 with the one or more convex surfaces 21 is formed as a unitary, one-piece member. The convex surfaces 21 may be integrally formed with the remainder of the receiver 20, or may be permanently attached to the receiver 20, such as by welding, adhesives, staking, press-fit, mechanical forming, and mechanical joining.

The profile of the channel 50 is designed to help facilitate the desired sliding motion between the receiver 20 and the rod 100. The profile discourages undesirable binding of the receiver 20 against the outer surface of rod 100. Further, the profile, in some embodiments, provides more material proximate the middle of channel 50, where clamping to the securing member 40 is most likely to occur. To further help facilitate the desired sliding motion, the channel 50 may include a suitable friction reducing material. In one embodiment, the channel 50 is coated with, or otherwise formed with, a suitable friction reducing material. For example, the interior surface may be coated with a low friction material (e.g., a ceramic or low friction polymer), and/or finished in a suitable manner such as anodizing and thermal diffusion coating, to reduce friction between the receiver 20 and the exterior surface of rod 100. In one embodiment, the receiver 20 is constructed from a suitable material such as cobalt chrome and PEEK. Alternatively, or additionally, the exterior surface of rod 100 may likewise be coated and/or finished.

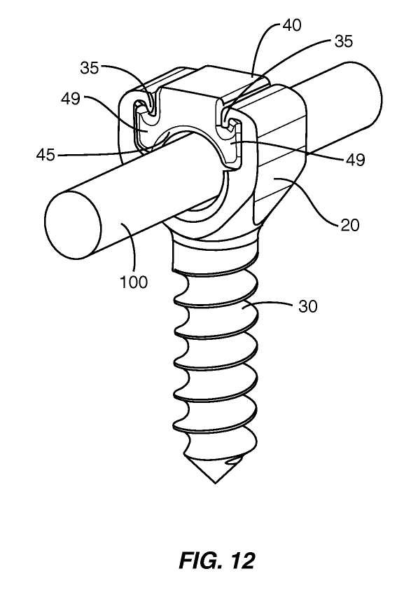

Receiver 20 may also include an opening 23 to receive the securing member 30. The opening 23 may be formed between separate side walls 25 as illustrated in FIGS. 1 and 12, or may extend through a side wall 25 as illustrated in FIGS. 5A and 5B. The opening 23 may be threaded, or may include one or more tabs 35 as illustrated in FIG. 12 that mate with the securing member 30. In one embodiment as illustrated in FIG. 5B, the opening 23 is positioned opposite from and aligned along the longitudinal axis 51 with an apex of the convex section 21.

In one embodiment used with in-line receivers 20, the securing member 40 fits within the opening 23 and includes a lower end 45 that extends into the channel 50 to contact the rod 100. In some embodiments, the lower end 45 includes a convex surface 41 that contacts against the rod 100. The securing member 40 may take any form known in the art, including a simple exteriorly threaded setscrew. FIG. 12 includes another embodiment with the securing member 40 including a pair of arms 49 that engage with the tabs 35 on the receiver 20. The securing member 40 may include a lower end 45 with a convex shape that provides for limited contact with the rod 100. In one embodiment, the securing member 40 extends into the channel 50 at a point directly opposite from the apex of the convex section 21.

In one embodiment as illustrated in FIG. 6, the securing member 40 is a threaded nut that engages with the fastener 30. The securing member 40 applies a compressive force to the side walls 25 to clamp the rod 100 within the receiver 20.

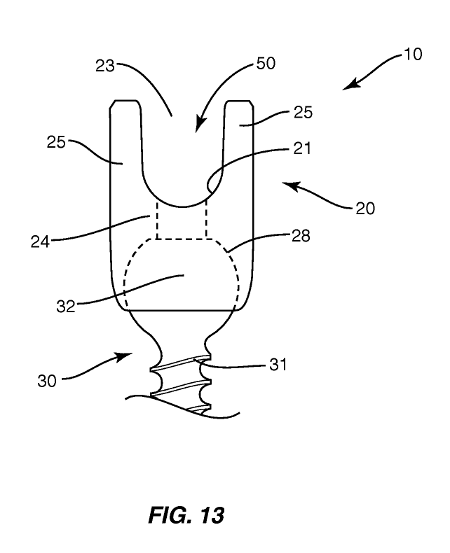

The fastener 30 includes a distal end that contacts with the vertebral member 200 and a proximal end that is operatively connected to the receiver 20. The fastener 30 may include a variety of configurations, including but not limited to a threaded shaft, screw, and hook. In one embodiment as illustrated in FIG. 5A and 5B, fastener 30 and receiver 20 are a unitary one-piece construction. In other embodiments, the fastener 3 and receiver 20 are separate elements. FIG. 6 illustrates an embodiment with the fastener 30 with an elongated shape with the distal end 31 being threaded to contact the vertebral member 200, and the proximal end 32 including a head sized to fit within the openings 26 of the receiver 20. FIG. 13 includes an embodiment with the fastener 30 including a screw with a head at the proximal end 32 that fits within a receptacle 28 formed in the base 24 of the receiver 20. This configuration allows for the receiver 20 to move about the head to position the channel 50 as necessary to receive the rod 100. Various other embodiments are disclosed in U.S. patent application Ser. No. 11/493,447 filed Jul. 26, 2006 and herein incorporated by reference.

Rod 100 may be made from a suitably strong rigid material known in the art, such as titanium, or from a semi-rigid material such as PEEK, polyurethane, polypropylene, or polyethylene. Rod 100 may include a variety of cross-sectional shapes including but not limited to circular, rectangular, square, and oval. Depending upon the context of use, the rod 100 may be linear or non-linear. The channel 50 is designed and the convex surface 21 tapered to accommodate the various shapes of the rod 100.

The stop 102 is secured to, or may be formed by, the corresponding end of rod 100. The stop 102 may take any form known in the art, such as a simple enlarged cap that is threaded onto the respective rod end. The stop 102 functions to prevent the anchor 10 from longitudinally moving off the rod 100 and maintaining the anchor 10 within a predetermined point that helps to limit the overall movement of the spinal segment being stabilized.

In one embodiment, the rod 100 does not include a stop 102. An end of the rod 100 may be positioned such that it should not escape from the anchor 10 under expected amounts of movement of the vertebral members 200.

The present invention may be carried out in other specific ways than those herein set forth without departing from the scope and essential characteristics of the invention. Further, the various aspects of the disclosed device and method may be used alone or in any combination, as is desired. The disclosed embodiments are, therefore, to be considered in all respects as illustrative and not restrictive, and all changes coming within the meaning and equivalency range of the appended claims are intended to be embraced therein.

What is claimed is:

1. An anchor to secure a rod to a vertebral member comprising:
a fastener including a distal end adapted to be connected to the vertebral member and a proximal end; and
a receiver operatively connected to the proximal end of the fastener, the receiver including a channel with a longitudinal axis and being sized to receive the rod, the receiver including a convex section that extends into the channel towards the longitudinal axis to contact the rod, the receiver and convex section being formed in a unitary one-piece construction.

2. The anchor of claim 1, wherein the fastener and the receiver are formed in a unitary one-piece construction.

3. The anchor of claim 1, wherein the channel is in-line with the fastener.

4. The anchor of claim 1, wherein the channel includes a substantially circular cross-sectional shape and the convex section extends around a majority of the channel.

5. The anchor of claim 1, wherein the receiver includes a second convex section that is spaced apart from the convex section, the second convex section extending towards the longitudinal axis to contact the rod.

6. The anchor of claim 1, further comprising a securing member adapted to connect to the receiver and including a lower edge with a convex shape that extends into the channel and is adapted to contact the rod.

7. The anchor of claim 1, wherein the receiver further includes a side wall that extends completely around the periphery of the channel.

8. The anchor of claim 1, wherein the receiver is movably connected to the proximal end of the fastener.

9. The anchor of claim 1, wherein the receiver includes a base and a pair of spaced-apart side walls with the channel formed therebetween.

10. An anchor to secure a rod to a vertebral member comprising:
a fastener including a distal end adapted to be connected to the vertebral member and a proximal end;
a receiver operatively connected to the proximal end of the fastener, the receiver including a channel sized to receive the rod with the channel including a longitudinal axis, the receiver further including a convex section that extends into the channel towards the longitudinal axis and an opening in communication with the channel and positioned opposite from and aligned along the longitudinal axis with an apex of the convex section, the receiver and convex section being formed in a unitary one-piece construction; and
a securing member sized to fit within the opening and including a lower edge adapted to extend into the channel and contact the rod.

11. The anchor of claim 10, wherein the convex section extends around a discrete section of the channel.

12. The anchor of claim 10, wherein the convex section extends continuously around a periphery of the channel.

13. The anchor of claim 10, wherein the receiver is in-line with the fastener.

14. The anchor of claim 10, wherein the convex section is centered at a midpoint of the channel.

15. The anchor of claim 10, further comprising a second convex section located axially along the longitudinal axis from the convex section.

16. An anchor to secure a rod to a vertebral member comprising:
a fastener including a distal end adapted to be connected to the vertebral member and a proximal end; and
a receiver operatively connected to the proximal end of the fastener and including a channel extending through the receiver such that the receiver extends around the channel, the channel including a longitudinal axis, the receiver further including a convex section that extends into the channel towards the longitudinal axis, the receiver and convex section being formed in a unitary one-piece construction.

17. The anchor of claim 16, further including a securing member sized to fit within an opening in the receiver and including a lower edge adapted to extend into the channel and contact the rod.

18. The anchor of claim 17, wherein the securing member and the convex section are centered at a midpoint of the longitudinal axis.

19. An anchor to secure a rod to a vertebral member comprising:
a fastener including a distal end adapted to be connected to the vertebral member and a proximal end; and
a receiver operatively connected to the proximal end of the fastener, the receiver including a channel sized to receive the rod with the channel including a longitudinal axis, the channel comprises a medial first section of reduced size that tapers both inwardly and outwardly relative to the longitudinal axis and respective end sections of relatively larger size, the receiver formed in a unitary one-piece construction.

20. The anchor of claim 19, further including a securing member that attaches to the receiver and includes a lower edge adapted to extend into the channel and contact the rod.

21. The anchor of claim 19, wherein said channel is defined by an interior wall that convexly curves toward said axis in said first section.
